(19) United States
(12) Patent Application Publication
(10) Pub. No.: US 2024/0307019 A1
(43) Pub. Date: Sep. 19, 2024

(54) X-Ray Image Acquisition Method and System, X-Ray Machine, and Storage Medium

(71) Applicant: Siemens Shanghai Medical Equipment Ltd., Shanghai (CN)

(72) Inventors: Yu Dan Wang, Shanghai (CN); Xiao Ai Fei, Shanghai (CN); You Sheng Yang, Shanghai (CN); Zhi Ming Yang, Shanghai (CN)

(73) Assignee: Siemens Shanghai Medical Equipment Ltd., Shanghai (CN)

(21) Appl. No.: 18/574,900

(22) PCT Filed: Jul. 12, 2022
(86) PCT No.: PCT/CN2022/105230

§ 371 Date: Dec. 28, 2023

(30) Foreign Application Priority Data
Jul. 13, 2021 (CN) 202110788415.X

(51) Int. Cl. A61B 6/00; G06T 5/50; G06T 7/00

(52) U.S. Cl. CPC A61B 6/5241; A61B 6/542; G06T 5/50; G06T 7/0012; G06T 2207/10116

(57) ABSTRACT

Techniques are disclosed for acquiring a captured target image of a target region. When it is determined that there is at least one to-be-optimized region of a bright region and/or a dark region in the target image, calculating, for each to-be-optimized region, a pixel average of the to-be-optimized region in the target image, and determining a to-be-optimized region received dose corresponding to the pixel average of the to-be-optimized region; adjusting an X-ray emission dose of an X-ray source according to a principle of making the to-be-optimized region received dose reach an X-ray reference received dose, and acquiring optimized images of the target region captured based on the X-ray emission dose adjusted to meet a requirement; and adding and synthesizing the optimized images, or adding and synthesizing the optimized images and the target image, to obtain an X-ray image of the target region.

### CROSS-REFERENCE TO RELATED APPLICATIONS

The present application is a U.S. National Stage entry of PCT Application no. PCT/CN2022/105230, filed Jul. 12, 2022, which claims priority to and the benefit of China patent application no. CN 202110788415.X, filed on Jul. 13, 2021, the contents of each of which are incorporated herein by reference in their entireties.

### TECHNICAL FIELD

The present disclosure relates to the medical field and, in particular, to an X-ray image acquisition method and system, an X-ray machine, and a computer-readable storage medium.

### BACKGROUND

In a medical angiography X-ray machine, an X-ray source and an X-ray receiver (for example, a flat panel detector) are installed opposite to each other so that X-rays generated by the X-ray source penetrate an object and are then incident on and detected by the X-ray receiver.

However, in practical applications, local image overexposure caused by large density differences of human tissues in a target region of interest is a common image quality problem, that is, an image part of low-density tissues or a thinner body part is overexposed, while an image part of high-density tissues or a thicker body part is underexposed. In this case, it is difficult to see all details simultaneously. For example, during an interventional treatment procedure, lower-density regions are often overexposed, such as lung or air overexposure, which results in larger clinical image contrast. Especially in a cardiac interventional operation, lung regions are overexposed more obviously, which affects the visibility of a stent, and a precise position of the stent cannot be acquired in the overexposed regions. In another example, when legs are scanned, the region between the legs in the image is overexposed. In still another example, when a cervical spine is scanned, both sides of the cervical spine are prone to overexposure.

FIG. 1 shows an X-ray image of thoracic and lumbar spines of a patient. As shown in FIG. 1, the thoracic spine part of the image is overexposed, while the lumbar spine part of the image is underexposed, making it difficult for doctors to simultaneously see the thoracic and lumbar spines clearly. However, often information is needed about both the thoracic and lumbar spines at the same time.

In this case, to deal with different filtering requirements, at present filters with different thicknesses are disposed between the X-ray source and the X-ray receiver to filter different amounts of X-rays so that different filters are switched to achieve ideal image requirements. In addition, those skilled in the art are still working to find other solutions.

### SUMMARY

In view of this, one aspect of embodiments of the present disclosure provides an X-ray image acquisition method, and other aspects provide an X-ray image acquisition system, an X-ray machine, and a computer-readable storage medium, which are used to acquire images with high imaging quality for human tissues with large density differences.

The X-ray image acquisition method provided in the embodiments of the present disclosure includes: acquiring a captured target image of a target region; when it is determined that there is at least one to-be-optimized region (also referred to herein as a sub-region or a candidate region) in the target image, calculating, for each to-be-optimized region, a pixel average of the to-be-optimized region in the target image; determining a to-be-optimized region received dose corresponding to the pixel average of the to-be-optimized region according to a relationship between a predetermined pixel average and an X-ray received dose of an X-ray receiver; adjusting an X-ray emission dose of an X-ray source according to a principle of making the to-be-optimized region received dose reach a predetermined X-ray reference received dose, and acquiring an optimized image (also referred herein to as an enhanced region) of the target region captured based on the X-ray emission dose adjusted to meet a requirement, where the at least one to-be-optimized region includes an overexposed bright region and/or an underexposed dark region; and adding and synthesizing at least one optimized image corresponding to the at least one to-be-optimized region, or adding and synthesizing at least one optimized image corresponding to the at least one to-be-optimized region and the target image, to obtain an X-ray image of the target region.

In an implementation, the at least one to-be-optimized region includes an overexposed bright region and an underexposed dark region; and the calculating, for each to-be-optimized region, a pixel average of the to-be-optimized region in the target image includes: calculating a histogram of the target image; finding a global threshold of the histogram by using a threshold segmentation method; and calculating, based on the global threshold, an average of pixels in the histogram with values less than the global threshold, to obtain a dark region pixel average; and calculating an average of pixels in the histogram with values greater than the global threshold, to obtain a bright region pixel average.

In an implementation, the adding and synthesizing at least one optimized image corresponding to the at least one to-be-optimized region, or adding and synthesizing at least one optimized image corresponding to the at least one to-be-optimized region and the target image includes: entirely superimposing the at least one optimized image corresponding to the at least one to-be-optimized region, or synthesizing image parts corresponding to respective to-be-optimized regions in optimized images corresponding to different to-be-optimized regions.

In an implementation, the at least one to-be-optimized region includes an overexposed bright region and/or an underexposed dark region; and the calculating, for each to-be-optimized region, a pixel average of the to-be-optimized region in the target image includes: calculating an average of pixels in the target image with brightness values greater than a first brightness threshold, to obtain a bright region pixel average; and/or calculating an average of pixels in the target image with brightness values less than a second brightness threshold, to obtain a dark region pixel average, where the second brightness threshold is less than or equal to the first brightness threshold.

In an implementation, the at least one to-be-optimized region includes an overexposed bright region and an underexposed dark region, and the second brightness threshold is equal to the first brightness threshold; and the adding and synthesizing at least one optimized image corresponding to the at least one to-be-optimized region, or adding and synthesizing at least one optimized image corresponding to the at least one to-be-optimized region and the target image includes: entirely superimposing the at least one optimized image corresponding to the at least one to-be-optimized region, or synthesizing image parts corresponding to respective to-be-optimized regions in optimized images corresponding to different to-be-optimized regions; or the at least one to-be-optimized region includes an overexposed bright region and an underexposed dark region, and the second brightness threshold is less than the first brightness threshold; or the at least one to-be-optimized region includes an overexposed bright region or an underexposed dark region, and either the bright region or the dark region is part of the target image; and the adding and synthesizing at least one optimized image corresponding to the at least one to-be-optimized region, or adding and synthesizing at least one optimized image corresponding to the at least one to-be-optimized region and the target image includes: entirely superimposing the at least one optimized image corresponding to the at least one to-be-optimized region and the target image, or synthesizing image parts corresponding to respective to-be-optimized regions in optimized images corresponding to different to-be-optimized regions and image parts in the target image other than the at least one to-be-optimized region.

In an implementation, the method further includes: detecting the pixels in the target image with the brightness values greater than the preset first brightness threshold to obtain overexposed pixels, and determining that there is an overexposed bright region in the target image when a quantity of the overexposed pixels reaches a preset first quantity threshold; and/or detecting the pixels in the target image with the brightness values less than the preset second brightness threshold to obtain underexposed pixels, and determining that there is a to-be-optimized underexposed dark region in the target image when a quantity of the underexposed pixels reaches a preset second quantity threshold, where the second brightness threshold is less than or equal to the first brightness threshold.

The X-ray image acquisition system provided in the embodiments of the present disclosure includes: a first unit configured to acquire a captured target image of a target region; a second unit configured to, when it is determined that there is at least one to-be-optimized region in the target image, calculate, for each to-be-optimized region, a pixel average of the to-be-optimized region in the target image; determine a to-be-optimized region received dose corresponding to the pixel average of the to-be-optimized region according to a relationship between a predetermined pixel average and an X-ray received dose of an X-ray receiver; and adjust an X-ray emission dose of an X-ray source according to a principle of making the to-be-optimized region received dose reach a predetermined X-ray reference received dose, and acquire an optimized image of the target region captured based on the X-ray emission dose adjusted to meet a requirement, where the at least one to-be-optimized region includes an overexposed bright region and/or an underexposed dark region; and a third unit, configured to add and synthesize at least one optimized image corresponding to the at least one to-be-optimized region, or add and synthesize at least one optimized image corresponding to the at least one to-be-optimized region and the target image, to obtain an X-ray image of the target region.

In an implementation, the at least one to-be-optimized region includes an overexposed bright region and an underexposed dark region; and the second unit is configured to calculate a histogram of the target image; find a global threshold of the histogram by using a threshold segmentation method; and calculate, based on the global threshold, an average of pixels in the histogram with values less than the global threshold, to obtain a dark region pixel average; and calculate an average of pixels in the histogram with values greater than the global threshold, to obtain a bright region pixel average.

In an implementation, the third unit is configured to entirely superimpose the at least one optimized image corresponding to the at least one to-be-optimized region, or synthesize image parts corresponding to respective to-be-optimized regions in optimized images corresponding to different to-be-optimized regions, to obtain the X-ray image of the target region.

In an implementation, the at least one to-be-optimized region includes an overexposed bright region and/or an underexposed dark region; and the second unit is configured to calculate an average of pixels in the target image with brightness values greater than a first brightness threshold, to obtain a bright region pixel average; and/or calculate an average of pixels in the target image with brightness values less than a second brightness threshold, to obtain a dark region pixel average.

In an implementation, the at least one to-be-optimized region includes the overexposed bright region and the underexposed dark region, and the second brightness threshold is equal to the first brightness threshold; and the third unit is configured to entirely superimpose the at least one optimized image corresponding to the at least one to-be-optimized region, or synthesize image parts corresponding to respective to-be-optimized regions in optimized images corresponding to different to-be-optimized regions, to obtain the X-ray image of the target region; or the at least one to-be-optimized region includes an overexposed bright region and an underexposed dark region, and the second brightness threshold is less than the first brightness threshold; or the at least one to-be-optimized region includes an overexposed bright region or an underexposed dark region, and either the bright region or the dark region is part of the target image; and the third unit is configured to entirely superimpose the at least one optimized image corresponding to the at least one to-be-optimized region and the target image, or synthesize image parts corresponding to respective to-be-optimized regions in optimized images corresponding to different to-be-optimized regions and image parts in the target image other than the at least one to-be-optimized region.

In an implementation, the method further includes: a fourth unit configured to detect the pixels in the target image with the brightness values greater than the preset first brightness threshold to obtain overexposed pixels, and determine that there is an overexposed bright region in the target image when a quantity of the overexposed pixels reaches a preset first quantity threshold; and/or detect the pixels in the target image with the brightness values less than the preset second brightness threshold to obtain underexposed pixels, and determine that there is an underexposed dark region in the target image when a quantity of the underexposed pixels reaches a preset second quantity threshold, where the second brightness threshold is less than or equal to the first brightness threshold.

Another X-ray image acquisition system provided in the embodiments of the present disclosure includes: at least one memory and at least one processor, where the at least one memory is configured to store a computer program; and the at least one processor is configured to call the computer program stored in the at least one memory to perform the X-ray image acquisition method according to any one of the foregoing implementations.

The X-ray machine provided in the embodiments of the present disclosure includes: the X-ray image acquisition system according to any one of the foregoing implementations.

The computer-readable storage medium provided in the embodiments of the present disclosure stores a computer program, which is executable by a processor to implement the X-ray image acquisition method according to any one of the foregoing implementations.

As can be seen from the foregoing solutions, in the embodiments of the present disclosure, when it is determined that there are to-be-optimized regions including an overexposed bright region and/or an underexposed dark region in the target image, for each to-be-optimized region, the X-ray emission dose is adjusted according to the X-ray received dose that meets the imaging quality requirement, the optimized images are acquired based on the X-ray emission dose adjusted to meet the requirement, and the optimized images are then added and synthesized to obtain the X-ray image that meets the imaging quality requirement as a whole.

In addition, whether there is a to-be-optimized region in the target image is automatically detected by the system, which can improve the intelligence and flexibility of the system application.

### BRIEF DESCRIPTION OF THE DRAWINGS

To enable a person of ordinary skill in the art to understand the foregoing and other features and advantages of the present disclosure more clearly, exemplary embodiments of the present disclosure are described in detail below with reference to the accompanying drawings. In the accompanying drawings:

FIG. 1 illustrates an X-ray image of thoracic and lumbar spines of a patient.

FIG. 2 illustrates an exemplary flowchart of an X-ray image acquisition method according to an embodiment of the present disclosure.

FIG. 3 illustrates a schematic diagram of a to-be-scanned body model in an example of the present disclosure.

FIG. 4A illustrates a schematic diagram of a target image in an example of the present disclosure.

FIG. 4B illustrates a schematic diagram of a histogram of the target image shown in FIG. 4A and a global threshold TS found based on the histogram.

FIG. 4C and FIG. 4D respectively illustrate schematic diagrams of a first optimized image and a second optimized image obtained by performing local image optimization based on the body model shown in FIG. 3.

FIG. 4E illustrates a schematic diagram of an X-ray image obtained by adding the first optimized image shown in FIG. 4C and the second optimized image shown in FIG. 4D.

FIG. 5 illustrates an exemplary structural diagram of an X-ray image acquisition system according to an embodiment of the present disclosure.

FIG. 6 illustrates an exemplary structural diagram of another X-ray image acquisition system according to an embodiment of the present disclosure.

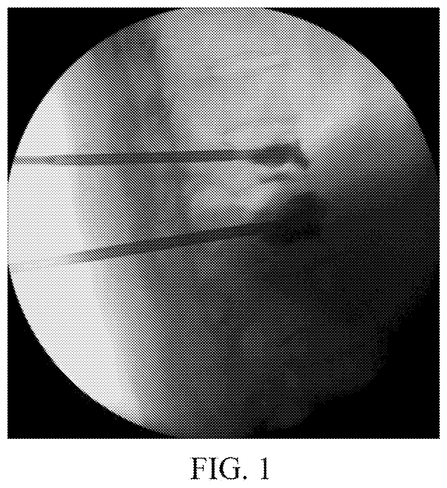

Reference numerals are as follows:

| Number | Meaning |
|---|---|
| S21 to S25 | Blocks |
| 31 | Pelvis model |
| 32 | Elbow model |
| 510 | First unit |
| 520 | Second unit |
| 530 | Third unit |
| 540 | Fourth unit |
| 61 | Memory |
| 62 | Processor |
| 63 | Display |
| 64 | Bus |

### DETAILED DESCRIPTION OF THE DISCLOSURE

In consideration of a limited dynamic range of an X-ray receiver, it is difficult to record all signals using a high signal-to-noise ratio. For a high absorption part of X-rays in human tissues, a dark region with high noise, that is, an underexposed region, may appear in an image; while for a low absorption part, a bright region, that is, an overexposed region, may appear in the image. Therefore, in the embodiments of the present disclosure, it is considered to obtain a final image by combining a plurality of images, and these images may be images optimized for different image parts. For example, when there are both a bright region and a dark region in images, one image may be focused on the dark region and the other on the bright region. The images are then added and synthesized to obtain a final image with good image quality, and the image can clearly present information about the dark region and the bright region.

For clearer understanding of the objectives, technical solutions, and effects of the present disclosure, specific implementations of the present disclosure are now illustrated with reference to the accompanying drawings, and same reference numerals in the accompanying drawings represent components with same structures or with similar structures and same functions.

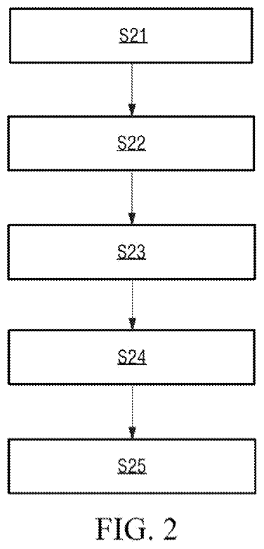

FIG. 2 is an exemplary flowchart of an X-ray image acquisition method according to an embodiment of the present disclosure. As shown in FIG. 2, the method may include the following process flow:

Block S21: Acquire a captured target image of a target region.

In this block, the target region may be a region of interest of a user in a clinical operation. In addition, the target region may be changed according to actual applications, and when the target region is changed, the image acquired in block S21 is also changed correspondingly.

During specific implementation, the solution in this embodiment may be performed in real time, and correspondingly, block S21 is also performed in real time; or the solution may be performed periodically, and correspondingly, block S21 is also performed periodically; or the solution may be performed by triggering a condition, and correspondingly, block S21 may be triggered when the target region is initially determined and when the target region is changed.

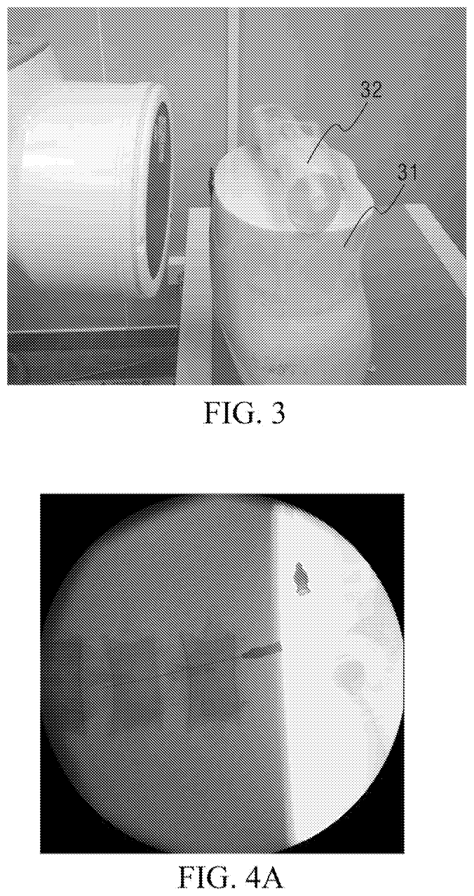

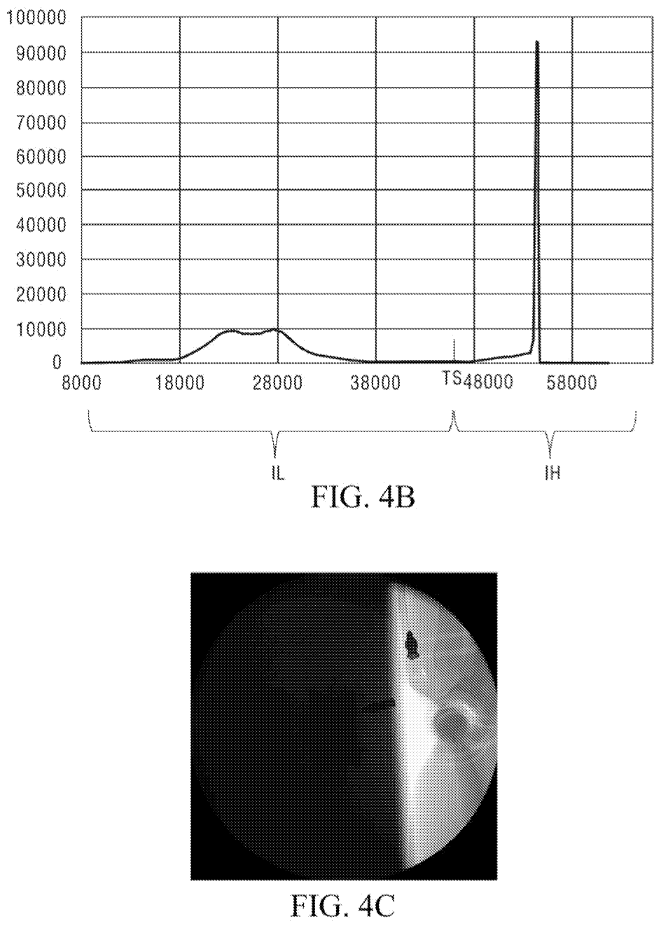

As shown in FIG. 3, a pelvis model 31 and an elbow model 32 are put together to simulate bodies with different thicknesses, thereby obtaining a schematic diagram of a target image shown in FIG. 4A. The pelvis is a high absorption part of X-rays, and the elbow is a low absorption part of X-rays.

Block S22: When it is determined that there is at least one to-be-optimized region such as an overexposed bright region and/or an underexposed dark region in the target image, calculate a pixel average such as a bright region pixel average and/or a dark region pixel average of the to-be-optimized region in the target image.

In this block, there may be various methods for determining whether there is an overexposed bright region and/or an underexposed dark region in the target image. For example, if the user can directly determine based on experience that there may be a to-be-optimized bright region and/or dark region in the image of the target region, the system can directly determine a to-be-optimized region, for example, according to an instruction or settings of the user. Correspondingly, in this embodiment, a pixel average such as a bright region pixel average and/or dark region pixel average of a to-be-optimized region in the target image can be directly calculated. For an application scenario in which the target region is constantly changed, or for the convenience of users with insufficient experience, or to improve the application flexibility of the method, a to-be-optimized region can be alternatively automatically determined by the system. That is, in this embodiment, before a pixel average such as a bright region pixel average and/or dark region pixel average of a to-be-optimized region in the target image is calculated, the method may further include: detecting whether there is a to-be-optimized region such as an overexposed bright region and/or an underexposed dark region in the target image. For example, during specific implementation, pixels in the target image with brightness values greater than a preset first brightness threshold can be detected based on a histogram technology, to obtain overexposed pixels, and it is determined that there is a to-be-optimized overexposed bright region in the target image when a quantity of the overexposed pixels reaches a preset first quantity threshold; otherwise it can be determined that there is no to-be-optimized overexposed bright region in the target image; and/or, pixels in the target image with brightness values less than a preset second brightness threshold are detected based on the histogram technology, to obtain underexposed pixels, and it is determined that there is a to-be-optimized underexposed dark region in the target image when a quantity of the underexposed pixels reaches a preset second quantity threshold; otherwise it can be determined that there is no to-be-optimized underexposed dark region in the target image. The second brightness threshold is less than or equal to the first brightness threshold. In addition, in other implementations, an image gradient calculation algorithm can be further used to determine a grayscale gradient distribution of a bright region and/or a dark region, an edge of the bright region and/or the dark region is further detected, and edge curves are then used to fit a bright region boundary and/or a dark region boundary.

There may be various methods for calculating a pixel average such as a bright region pixel average and/or a dark region pixel average of a to-be-optimized region in the target image.

For example, if it is determined that there are both an overexposed bright region and an underexposed dark region to be optimized in the target image, in a first example, a histogram of the target image can be calculated first, and a global threshold of the histogram can be found by using a threshold segmentation method such as a maximum between-class variance method (also referred to as the OSTU method). FIG. 4B is a schematic diagram of a histogram of the target image shown in FIG. 4A and a global threshold TS found based on the histogram. An average of pixels IH in the histogram with values greater than the global threshold can be then calculated based on the global threshold, to obtain a bright region pixel average; and an average of pixels IL in the histogram with values less than the global threshold can be calculated, to obtain a dark region pixel average.

In a second example, an average of pixels in the target image with brightness values greater than a first brightness threshold can be calculated based on the histogram technology, to obtain a bright region pixel average; and an average of pixels in the target image with brightness values less than a second brightness threshold can be calculated, to obtain a dark region pixel average. The second brightness threshold is less than or equal to the first brightness threshold.

If it is determined that there is a to-be-optimized overexposed bright region in the target image, but there is no underexposed dark region, or even if there is a dark region, but the optimization on the dark region is not considered, an average of pixels in the target image with brightness values greater than a first brightness threshold can be calculated based on the histogram technology, to obtain a bright region pixel average.

If it is determined that there is a to-be-optimized underexposed dark region in the target image, but there is no overexposed bright region, or even if there is a bright region, but the optimization on the bright region is not considered, an average of pixels in the target image with brightness values less than a second brightness threshold can be calculated based on the histogram technology, to obtain a dark region pixel average.

Certainly, when it is determined that there is no overexposed bright region or underexposed dark region to be optimized in the target image, the target image can be directly used as an X-ray image of the target region.

Block S23: Determine a to-be-optimized region received dose corresponding to the pixel average of the to-be-optimized region according to a relationship between a predetermined pixel average and an X-ray received dose of an X-ray receiver, for example, determine a bright region received dose corresponding to the bright region pixel average and/or a dark region received dose corresponding to the dark region pixel average.

In this block, if there are both an overexposed bright region and an underexposed dark region to be optimized in the target image, a bright region received dose corresponding to the bright region pixel average and a dark region received dose corresponding to the dark region pixel average are determined according to a relationship between a predetermined pixel average and an X-ray received dose of an X-ray receiver.

If there is a to-be-optimized overexposed bright region in the target image, but there is no underexposed dark region, or even if there is a dark region, but the optimization on the dark region is not considered, a bright region received dose corresponding to the bright region pixel average is determined according to a relationship between a predetermined pixel average and an X-ray received dose of an X-ray receiver.

If there is a to-be-optimized underexposed dark region in the target image, but there is no overexposed bright region, or even if there is a bright region, but the optimization on the bright region is not considered, a dark region received dose corresponding to the dark region pixel average is determined according to a relationship between a predetermined pixel average and an X-ray received dose of an X-ray receiver.

Block S24: Compare the to-be-optimized region received dose with a predetermined X-ray reference received dose that meets an imaging requirement, adjust an X-ray emission dose of an X-ray source according to a principle of making the to-be-optimized region received dose reach the X-ray reference received dose, and acquire an optimized image of the target region captured based on the X-ray emission dose adjusted to meet the requirement. For example, the bright region received dose is compared with a predetermined X-ray reference received dose that meets an imaging requirement, an X-ray emission dose of an X-ray source is adjusted according to a principle of making the bright region received dose reach the X-ray reference received dose or close to the X-ray reference received dose by a preset degree, and a first optimized image of the target region captured based on the X-ray emission dose adjusted to meet the requirement is acquired; and/or, the dark region received dose is compared with the X-ray reference received dose, an X-ray emission dose of an X-ray source is adjusted according to a principle of making the dark region received dose reach the X-ray reference received dose or close to the X-ray reference received dose by a preset degree, and a second optimized image of the target region captured based on the X-ray emission dose adjusted to meet the requirement is acquired.

In this embodiment, the X-ray reference received dose may be a value range, such as A 10% or A 5%, where A is the predetermined X-ray received dose that meets the imaging requirement, for example, 100.

The process of adjusting an X-ray emission dose of an X-ray source according to a principle of making the to-be-optimized region received dose reach the X-ray reference received dose may be a cyclic process, which is because one adjustment may not guarantee that the corresponding X-ray received dose can reach the X-ray reference received dose. Therefore, after one adjustment is performed, a current adjusted image of the target region captured based on the adjusted X-ray emission dose can be acquired, and a pixel average of a to-be-optimized region in the current adjusted image can be calculated. A to-be-optimized region received dose corresponding to the pixel average of the to-be-optimized region is determined according to a relationship between the pixel average and an X-ray received dose of an X-ray receiver. Whether the to-be-optimized region received dose reaches the X-ray reference received dose is determined, and if yes, the current adjusted image is used as an optimized image corresponding to the to-be-optimized region; otherwise the X-ray emission dose of the X-ray source is adjusted again, and the process returns to the block of acquiring a current adjusted image of the target region captured based on the adjusted X-ray emission dose. The adjustment of the X-ray emission dose of the X-ray source can be implemented by adjusting parameters such as voltage and current of the X-ray source.

In this block, if there are both an overexposed bright region and an underexposed dark region to be optimized in the target image, a first optimized image and a second optimized image are acquired. FIG. 4C and FIG. 4D are respectively schematic diagrams of a first optimized image and a second optimized image obtained by performing local image optimization based on the body model shown in FIG. 3.

If there is a to-be-optimized overexposed bright region in the target image, but there is no underexposed dark region, or even if there is a dark region, but the optimization on the dark region is not considered, only the first optimized image is acquired.

If there is a to-be-optimized underexposed dark region in the target image, but there is no overexposed bright region, or even if there is a bright region, but the optimization of the bright region is not considered, only the second optimized image is acquired.

Block S25: Add and synthesize at least one optimized image corresponding to the at least one to-be-optimized region, or add and synthesize at least one optimized image corresponding to the at least one to-be-optimized region and the target image, to obtain an X-ray image of the target region. For example, the first optimized image and the second optimized image are added and synthesized, or the first optimized image and/or the second optimized image and the target image are added and synthesized, to obtain an X-ray image of the target region.

In this block, if there are both an overexposed bright region and an underexposed dark region to be optimized in the target image, and a bright region pixel average and a dark region pixel average are acquired by using the method in the first example in block S22, the first optimized image and the second optimized image can be directly added and synthesized to obtain an X-ray image of the target region. Specifically, the at least one optimized image corresponding to the at least one to-be-optimized region can be entirely superimposed. FIG. 4E shows an X-ray image obtained by entirely superimposing the first optimized image shown in FIG. 4C and the second optimized image shown in FIG. 4D. It can be seen that the optimized and recombined image is a clear X-ray image that can present tissues with large density differences. Certainly, in other implementations, image parts corresponding to respective to-be-optimized regions in optimized images corresponding to different to-be-optimized regions can be alternatively synthesized. For example, an image part corresponding to a to-be-optimized region in an optimized image can be segmented based on the bright region boundary and/or the dark region boundary, and the segmented parts are synthesized to obtain an X-ray image of the target region.

If there are both an overexposed bright region and an underexposed dark region to be optimized in the target image, and a bright region pixel average and a dark region pixel average are acquired by using the method in the second example in block S22, but the second brightness threshold is equal to the first brightness threshold, the first optimized image and the second optimized image can still be directly added and synthesized as described above to obtain an X-ray image of the target region. If a bright region pixel average and a dark region pixel average are acquired by using the method in the second example in block S22, but the second brightness threshold is less than the first brightness threshold, the first optimized image, the second optimized image, and the target image can be added and synthesized to obtain an X-ray image of the target region.

If there is a to-be-optimized overexposed bright region in the target image, but there is no underexposed dark region, or even if there is a dark region, but the optimization on the dark region is not considered, the first optimized image and the target image can be added and synthesized to obtain an X-ray image of the target region.

If there is a to-be-optimized underexposed dark region in the target image, but there is no overexposed bright region, or even if there is a bright region, but the optimization on the bright region is not considered, the second optimized image and the target image can be added and synthesized to obtain an X-ray image of the target region.

The adding and synthesizing at least one optimized image corresponding to the at least one to-be-optimized region and the target image may be entirely superimposing the at least one optimized image corresponding to the at least one to-be-optimized region and the target image, or synthesizing image parts corresponding to respective to-be-optimized regions in optimized images corresponding to different to-be-optimized regions and image parts in the target image other than the at least one to-be-optimized region. For example, an image part corresponding to a to-be-optimized region in an optimized image and an image part in the target image other than the at least one to-be-optimized region can be segmented based on the bright region boundary and/or the dark region boundary, and the segmented parts are then synthesized to obtain an X-ray image of the target region.

The foregoing blocks S21 to S25 in this embodiment are not used to limit the execution order of the technical features. For example, if there are both an overexposed bright region and an underexposed dark region to be optimized in the target image, in addition to the execution order shown in block S21 to block S25 in FIG. 2, the execution order in this embodiment may be alternatively: calculating a bright region pixel average in the target image; determining a bright region received dose corresponding to the bright region pixel average according to a relationship between a predetermined pixel average and an X-ray received dose of an X-ray receiver; and comparing the bright region received dose with a predetermined X-ray reference received dose that meets an imaging requirement, adjusting an X-ray emission dose of an X-ray source according to a principle of making the bright region received dose reach the X-ray reference received dose or close to the X-ray reference received dose by a preset degree, and acquiring a first optimized image of the target region captured based on the adjusted X-ray emission dose; calculating a dark region pixel average in the target image; determining a dark region received dose corresponding to the dark region pixel average according to a relationship between a predetermined pixel average and an X-ray received dose of an X-ray receiver; and comparing the dark region received dose with an X-ray reference received dose, adjusting an X-ray emission dose of an X-ray source according to a principle of making the dark region received dose reach the X-ray reference received dose or close to the X-ray reference received dose by a preset degree, and acquiring a second optimized image of the target region captured based on the adjusted X-ray emission dose. In addition, the execution order may be alternatively: calculating a dark region pixel average in the target image; determining a dark region received dose corresponding to the dark region pixel average according to a relationship between a predetermined pixel average and an X-ray received dose of an X-ray receiver; and comparing the dark region received dose with an X-ray reference received dose, adjusting an X-ray emission dose of an X-ray source according to a principle of making the dark region received dose reach the X-ray reference received dose or close to the X-ray reference received dose by a preset degree, and acquiring a second optimized image of the target region captured based on the adjusted X-ray emission dose; calculating a bright region pixel average in the target image; determining a bright region received dose corresponding to the bright region pixel average according to a relationship between a predetermined pixel average and an X-ray received dose of an X-ray receiver; and comparing the bright region received dose with the predetermined X-ray reference received dose that meets an imaging requirement, adjusting an X-ray emission dose of an X-ray source according to a principle of making the bright region received dose reach the X-ray reference received dose or close to the X-ray reference received dose by a preset degree, and acquiring a first optimized image of the target region captured based on the adjusted X-ray emission dose. The specific execution order can be adjusted according to actual requirements, and is not limited herein.

The X-ray image acquisition method in the embodiments of the present disclosure is described in detail above, and an X-ray image acquisition system in the embodiments of the present disclosure is described in detail below. The X-ray image acquisition system in the embodiments of the present disclosure may be used to implement the X-ray image acquisition method in the embodiments of the present disclosure. For details not disclosed in detail in the system embodiments of the present disclosure, refer to the corresponding descriptions in the method embodiments of the present disclosure, and the details are not repeated herein.

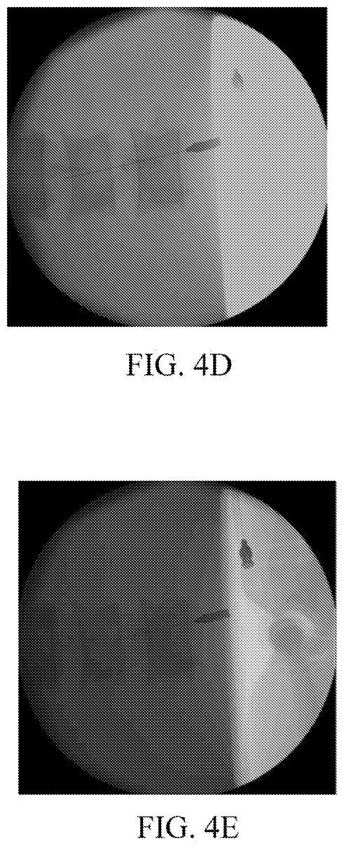

FIG. 5 illustrates an exemplary structural diagram of an X-ray image acquisition system according to an embodiment of the present disclosure. As shown in the solid-line parts in FIG. 5, the system may include: a first unit 510, a second unit 520, and a third unit 530. Each of the first unit, the second unit, the third unit, and the fourth unit (which is discussed in further detail below) may be implemented as any suitable processor and/or processing circuitry configured to perform their respective functions, which may include, for example, via the execution of instructions stored in a suitable memory. Thus, the first unit, the second unit, the third unit, and the fourth unit may alternatively be referred to as stated herein.

The first unit 510 (also referred to as image acquisition processing circuitry, an image acquisition unit, or an image acquirer) is configured to acquire a captured target image of a target region.

The second unit 520 (also referred to as computational processing circuitry, determination processing circuitry, a determination unit, or a determiner) is configured to, when it is determined that there is at least one to-be-optimized region in the target image, calculate, for each to-be-optimized region, a pixel average of the to-be-optimized region in the target image; determine a to-be-optimized region received dose corresponding to the pixel average of the to-be-optimized region according to a relationship between a predetermined pixel average and an X-ray received dose of an X-ray receiver; and adjust an X-ray emission dose of an X-ray source according to a principle of making the to-be-optimized region received dose reach a predetermined X-ray reference received dose, and acquire an optimized image of the target region captured based on the X-ray emission dose adjusted to meet a requirement, where the at least one to-be-optimized region includes an overexposed bright region and/or an underexposed dark region. For example, when it is determined that there is a to-be-optimized overexposed bright region in the target image, a bright region pixel average in the target image is calculated; a bright region received dose corresponding to the bright region pixel average is determined according to a relationship between a predetermined pixel average and an X-ray received dose of an X-ray receiver; and the bright region received dose is compared with a predetermined X-ray reference received dose that meets an imaging requirement, an X-ray emission dose of an X-ray source is adjusted according to a principle of making the bright region received dose reach the X-ray reference received dose, and a first optimized image of the target region captured based on the X-ray emission dose adjusted to meet the requirement is acquired; and/or, when it is determined that there is a to-be-optimized underexposed dark region in the target image, a dark region pixel average in the target image is calculated; a dark region received dose corresponding to the dark region pixel average is determined according to a relationship between a predetermined pixel average and an X-ray received dose of an X-ray receiver; and the dark region received dose is compared with the X-ray reference received dose, an X-ray emission dose of an X-ray source is adjusted according to a principle of making the dark region received dose reach the X-ray reference received dose, and a second optimized image of the target region captured based on the X-ray emission dose adjusted to meet the requirement is acquired.

Corresponding to the method shown in FIG. 2, when it is determined that there are an overexposed bright region and an underexposed dark region to be optimized in the target image, the second unit 520 may perform the method in the first example in block S22: calculating a histogram of the target image; finding a global threshold of the histogram by using a threshold segmentation method such as the OSTU method; and calculating, based on the global threshold, an average of pixels in the histogram with values less than the global threshold, to obtain a dark region pixel average; and calculating an average of pixels in the histogram with values greater than the global threshold, to obtain a bright region pixel average. Alternatively, when it is determined that there are an overexposed bright region and/or an underexposed dark region to be optimized in the target image, the second unit 520 may perform the method in the second example in block S22: calculating an average of pixels in the target image with brightness values greater than a first brightness threshold based on a histogram technology, to obtain a bright region pixel average; and/or calculating an average of pixels in the target image with brightness values less than a second brightness threshold based on the histogram technology, to obtain a dark region pixel average.

The third unit 530 (also referred to as image synthesizing processing circuitry, an image synthesizing unit, or an adder) is configured to add and synthesize at least one optimized image corresponding to the at least one to-be-optimized region, or add and synthesize at least one optimized image corresponding to the at least one to-be-optimized region and the target image, to obtain an X-ray image of the target region. For example, the first optimized image and the second optimized image are added, or the first optimized image and/or the second optimized image and the target image are added, to obtain an X-ray image of the target region.

Corresponding to the method shown in FIG. 2, when it is determined that there are an overexposed bright region and an underexposed dark region to be optimized in the target image, and the second unit 520 performs the method in the first example in block S22 to calculate a pixel average of the to-be-optimized region and further obtain an optimized image, or when the second unit 520 performs the method in the second example in block S22 to calculate a pixel average of the to-be-optimized region and further obtain an optimized image, and the second brightness threshold is equal to the first brightness threshold, the third unit 530 may entirely superimpose the at least one optimized image corresponding to the at least one to-be-optimized region, or synthesize image parts corresponding to respective to-be-optimized regions in optimized images corresponding to different to-be-optimized regions, to obtain an X-ray image of the target region. When it is determined that there are an overexposed bright region and an underexposed dark region to be optimized in the target image, and the second unit 520 performs the method in the second example in block S22 to calculate a pixel average of the to-be-optimized region and further obtain an optimized image, if the second brightness threshold is less than the first brightness threshold, or when it is determined that there is only an overexposed bright region or an underexposed dark region to be optimized in the target image, the third unit 530 may entirely superimpose the at least one optimized image corresponding to the at least one to-be-optimized region and the target image, or synthesize image parts corresponding to respective to-be-optimized regions in optimized images corresponding to different to-be-optimized regions and image parts in the target image other than the at least one to-be-optimized region, to obtain an X-ray image of the target region.

Corresponding to the method shown in FIG. 2, the system in the embodiments of the present disclosure may be shown in the dotted-line part in FIG. 5, and further includes: a fourth unit 540 (also referred to as pixel detection processing circuitry, a pixel detection unit, or a pixel detector), configured to detect, based on the histogram technology, the pixels in the target image with the brightness values greater than the preset first brightness threshold to obtain overexposed pixels, and determine that there is a to-be-optimized overexposed bright region in the target image when a quantity of the overexposed pixels reaches a preset first quantity threshold; and/or detect, based on the histogram technology, the pixels in the target image with the brightness values less than the preset second brightness threshold to obtain underexposed pixels, and determine that there is a to-be-optimized underexposed dark region in the target image when a quantity of the underexposed pixels reaches a preset second quantity threshold, where the second brightness threshold is less than or equal to the first brightness threshold.

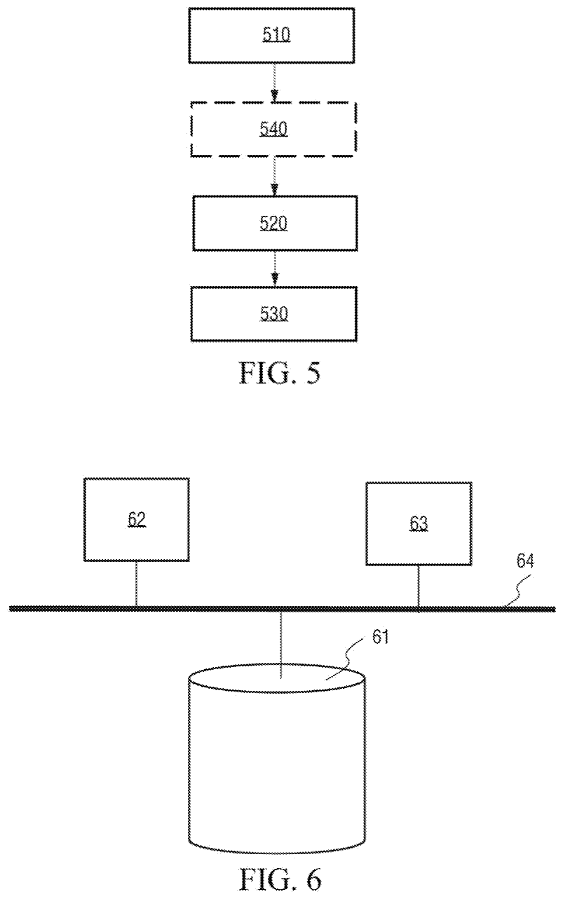

FIG. 6 is a schematic structural diagram of another X-ray image acquisition system according to an embodiment of the present disclosure. As shown in FIG. 6, the system may include: at least one memory 61, at least one processor 62, and at least one display 63. In addition, some other components such as a communication port may be further included. These components communicate by using a bus 64.

The at least one memory 61 is configured to store a computer program. In an implementation, the computer program may be understood as including various modules of the X-ray image acquisition system shown in FIG. 5. In addition, the at least one memory 61 may further store an operating system and the like. The operating system includes but is not limited to: an Android operating system, a Symbian operating system, a Windows operating system, a Linux operating system, and the like.

The at least one display 63 is configured to display a captured target image, a first optimized image and/or a second optimized image, a final X-ray image, and the like.

The at least one processor 62 is configured to call the computer program stored in the at least one memory 61 to perform the X-ray image acquisition method according to the embodiments of the present disclosure. The processor 62 may be a CPU, a processing unit/module, an ASIC, a logical module, a programmable gate array, or the like. The processor may receive and transmit data through the communication port.

An embodiment of the present disclosure further provides an X-ray machine, which includes the X-ray image acquisition system according to any one of the foregoing implementations.

It should be noted that, not all blocks and modules in the procedures and the structural diagrams are necessary, and some blocks or modules may be omitted according to an actual requirement. An execution order of the blocks is not fixed and may be adjusted according to requirements. Division of the modules is merely functional division for ease of description. During actual implementation, one module may be implemented separately by a plurality of modules, and functions of the plurality of modules may alternatively be implemented by the same module. The modules may be located in the same device or in different devices.

It can be understood that hardware modules in the implementations may be implemented in a mechanic manner or an electronic manner. For example, a hardware module may include specially designed permanent circuits or logic devices (for example, an application-specific processor such as an FPGA or an ASIC) to complete specific operations. The hardware module may also include temporarily configured programmable logic devices or circuits (for example, including a universal processor or another programmable processor) to perform specific operations. The hardware module is implemented by specifically using the mechanical manner, using the application-specific permanent circuits, or using the temporarily configured circuits (for example, configured by software), which can be decided according to consideration of costs and time.

In addition, an embodiment of the present disclosure further provides a computer-readable storage medium, storing a computer program, where the computer program is executable by a processor to implement the X-ray image acquisition method according to the embodiments of the present disclosure. Specifically, a system or an apparatus that is equipped with a storage medium may be provided. The storage medium stores software program code that implements functions of any implementation in the foregoing embodiments, and a computer (a CPU or an MPU) of the system or the apparatus is enabled to read and execute the program code stored in the storage medium. In addition, a program code based instruction may also be used to enable an operating system or the like running in the computer to complete some or all actual operations. The program code read from the storage medium may also be written into a memory that is disposed in an expansion board inserted in the computer, or may be written into a memory that is disposed in an expansion unit connected to the computer, and then a CPU or the like that is installed on the expansion board or expansion unit may be enabled to execute some or all actual operations based on the instructions of the program code, so as to implement the functions of any one of the foregoing implementations. Implementations of the storage medium for providing the program code may include a floppy disk, a hard disk, a magneto-optical disk, an optical memory (such as a CD-ROM, a CD-R, a CD-RW, a DVD-ROM, a DVD-RAM, a DVD-RW, and a DVD+RW), a magnetic tape, a non-volatile storage card, and a ROM. Optionally, the program code may be downloaded from a server computer by using a communication network.

As can be seen from the foregoing solutions, in the embodiments of the present disclosure, when it is determined that there are to-be-optimized regions including an overexposed bright region and/or an underexposed dark region in the target image, for each to-be-optimized region, the X-ray emission dose is adjusted according to the X-ray received dose that meets the imaging quality requirement, the optimized images are acquired based on the X-ray emission dose adjusted to meet the requirement, and the optimized images are then added and synthesized to obtain the X-ray image that meets the imaging quality requirement as a whole.

In addition, whether there is a to-be-optimized region in the target image is automatically detected by the system, which can improve the intelligence and flexibility of the system application.

In this specification, “exemplary” and “schematic” indicate “serving as an example, a case, or description”, and any illustration or implementation described as “exemplary” and “schematic” in this specification should not be interpreted as a more preferred or more advantageous technical solution.

For brevity of the accompanying drawings, only parts related to the present disclosure are schematically shown in the accompanying drawings, and do not represent an actual structure as a product.

In this specification, “one” not only indicates “only one”, but also indicates “more than one”. In this specification, “first”, “second”, and the like are only used to distinguish from each other, but not to indicate, an order, and the like.

The foregoing descriptions are merely exemplary embodiments of the present disclosure, and are not intended to limit the present disclosure. Any modification, equivalent replacement, or improvement made within the spirit and principle of the present disclosure shall fall within the protection scope of the present disclosure.

1.-15. (canceled)

16. An X-ray image acquisition method, comprising:
acquiring a captured target image of a target region;
when it is determined that there is at least one candidate region in the target image, for each candidate region:
calculating an average of pixel values in the candidate region;
determining a received dose corresponding to the average of pixel values in the candidate region according to a relationship between a predetermined pixel value average and an X-ray received dose of an X-ray receiver;
adjusting an X-ray emission dose of an X-ray source based upon the received dose reaching a predetermined X-ray reference received dose;
acquiring an enhanced image of the target region based on the X-ray emission dose being adjusted to meet the predetermined X-ray reference received dose,
wherein the at least one candidate region comprises an overexposed region and/or an underexposed region; and
adding and synthesizing at least one enhanced image corresponding to (i) the at least one candidate region, or (ii) the at least one candidate region and the target image, to obtain an X-ray image of the target region.

17. The X-ray image acquisition method according to claim 16, wherein the at least one candidate region comprises an overexposed region and an underexposed region, and wherein calculating the average of pixel values of each candidate region comprises:
calculating a histogram of the target image;
identifying a global threshold value of the histogram using threshold segmentation;
calculating, based on the global threshold, an average of pixel values in the histogram having values less than the global threshold to obtain a dark region pixel average; and
calculating an average of pixel values in the histogram having values greater than the global threshold value to obtain a bright region pixel average.

18. The X-ray image acquisition method according to claim 16, wherein the adding and synthesizing the at least one enhanced image comprises:
entirely superimposing the at least one enhanced image corresponding to the at least one candidate region, or synthesizing image parts corresponding to respective candidate regions in enhanced images corresponding to different candidate regions, to obtain the X-ray image of the target region.

19. The X-ray image acquisition method according to claim 16, wherein the at least one candidate region comprises an overexposed region and/or an underexposed region, and
wherein the calculating of the average pixel values of each candidate region comprises:
calculating an average of pixel values in the target image having brightness values greater than a first brightness threshold value to obtain a bright region pixel average; and/or
calculating an average of pixel values in the target image having brightness values less than a second brightness threshold value to obtain a dark region pixel average,
wherein the second brightness threshold value is less than or equal to the first brightness threshold value.

20. The X-ray image acquisition method according to claim 19, wherein:
the at least one candidate region comprises an overexposed region and an underexposed region,
the second brightness threshold value is equal to the first brightness threshold value, and
the adding and synthesizing the at least one enhanced image comprises:
entirely superimposing the at least one enhanced image corresponding to the at least candidate region, or synthesizing image parts corresponding to respective candidate regions in enhanced images corresponding to different candidate regions.

21. The X-ray image acquisition method according to claim 19, wherein:
the at least one candidate region comprises an overexposed region and an underexposed region,
the second brightness threshold value is less than the first brightness threshold value, and
the overexposed region or the underexposed region is part of the target image; and
the adding and synthesizing the at least one enhanced image comprises:
(i) entirely superimposing the at least one enhanced image corresponding to the at least one candidate region and the target image, or (ii) synthesizing image parts corresponding to respective candidate regions in enhanced images corresponding to different candidate regions and image parts in the target image other than the at least one candidate region.

22. The X-ray image acquisition method according to claim 16, further comprising:
(i) detecting pixels in the target image having brightness values greater than a predetermined first brightness threshold value to obtain overexposed pixels, and/or (ii) detecting pixels in the target image having brightness values less than a predetermined second brightness threshold value to obtain underexposed pixels; and
(i) identifying, in response to a determination that a quantity of the overexposed pixels is equal to a predetermined first quantity threshold value, an overexposed region in the target image, and/or (ii) identifying, in response to a determination a quantity of the underexposed pixels in the target image when a reaches a preset second quantity threshold, an underexposed region in the target image,
wherein the second brightness threshold value is less than or equal to the first brightness threshold value.

23. An X-ray image acquisition system, comprising:
image acquisition processing circuitry configured to acquire a captured target image of a target region;
computational processing circuitry configured to, when it is determined that there is at least one candidate region in the target image, for each candidate region:
calculate an average of pixel values in the candidate region;
determine a received dose corresponding to the average of pixel values in the candidate region according to a relationship between a predetermined pixel value average and an X-ray received dose of an X-ray receiver;
adjust an X-ray emission dose of an X-ray source based upon the received dose reaching a predetermined X-ray reference received dose; and
acquire an enhanced image of the target region based on the X-ray emission dose being adjusted to meet the predetermined X-ray reference received dose,
wherein the at least one candidate region comprises an overexposed region and/or an underexposed region; and
image synthesizing processing circuitry configured to add and synthesize at least one enhanced image corresponding to (i) the at least one candidate region, or (ii) the at least one candidate region and the target image, to obtain an X-ray image of the target region.

24. The X-ray image acquisition system according to claim 23,
wherein the at least one candidate region comprises an overexposed region and an underexposed region, and
wherein the computational processing circuitry is configured to calculate the average of pixel values of each candidate region by:
calculating a histogram of the target image;
identifying a global threshold value of the histogram using threshold segmentation;
calculating, based on the global threshold, an average of pixel values in the histogram having values less than the global threshold to obtain a dark region pixel average; and
calculating an average of pixel values in the histogram having values greater than the global threshold value to obtain a bright region pixel average.

25. The X-ray image acquisition system according to claim 23, wherein the image synthesizing processing circuitry is configured to entirely superimpose the at least one enhanced image corresponding to the at least one candidate region, or synthesize image parts corresponding to respective candidate regions in enhanced images corresponding to different candidate regions, to obtain the X-ray image of the target region.

26. The X-ray image acquisition system according to claim 23, wherein the at least one candidate region comprises an overexposed region and/or an underexposed region, and
wherein the computational processing circuitry is configured to calculating the average pixel values of each candidate region by:
calculating an average of pixel values in the target image having brightness values greater than a first brightness threshold value to obtain a bright region pixel average; and/or
calculating an average of pixel values in the target image having brightness values less than a second brightness threshold value to obtain a dark region pixel average, and
wherein the second brightness threshold value is less than or equal to the first brightness threshold value.

27. The X-ray image acquisition system according to claim 26, wherein:
the at least one candidate region comprises an overexposed region and an underexposed region,
the second brightness threshold value is equal to the first brightness threshold value, and
the image synthesizing processing circuitry is configured to add and synthesize the at least one enhanced image by entirely superimposing the at least one enhanced image corresponding to the at least candidate region, or synthesizing image parts corresponding to respective candidate regions in enhanced images corresponding to different candidate regions.

28. The X-ray image acquisition system according to claim 26, wherein:
the at least one candidate region comprises an overexposed region and an underexposed region,
the second brightness threshold value is less than the first brightness threshold value, and the overexposed region or the underexposed region is part of the target image; and
the image synthesizing processing circuitry is configured to add and synthesize the at least one enhanced image by (i) entirely superimposing the at least one enhanced image corresponding to the at least one candidate region and the target image, or (ii) synthesizing image parts corresponding to respective candidate regions in enhanced images corresponding to different candidate regions and image parts in the target image other than the at least one candidate region.

29. The X-ray image acquisition system according to claim 23, further comprising:
pixel detection processing circuitry configured to:
(i) detect pixels in the target image having brightness values greater than a predetermined first brightness threshold value to obtain overexposed pixels, and/or (ii) detect pixels in the target image having brightness values less than a predetermined second brightness threshold value to obtain underexposed pixels; and
(i) identify, in response to a determination that a quantity of the overexposed pixels is equal to a predetermined first quantity threshold value, an overexposed region in the target image, and/or (ii) identify, in response to a determination a quantity of the underexposed pixels in the target image when a reaches a preset second quantity threshold, an underexposed region in the target image,
wherein the second brightness threshold value is less than or equal to the first brightness threshold value.

30. A computer-readable storage medium having instructions stored thereon that, when executed by a processor of an X-ray image acquisition system, cause the X-ray image acquisition system to:
acquire a captured target image of a target region;
when it is determined that there is at least one candidate region in the target image, for each candidate region:
calculate an average of pixel values in the candidate region;
determine a received dose corresponding to the average of pixel values in the candidate region according to a relationship between a predetermined pixel value average and an X-ray received dose of an X-ray receiver;
adjust an X-ray emission dose of an X-ray source based upon the received dose reaching a predetermined X-ray reference received dose;
acquire an enhanced image of the target region based on the X-ray emission dose being adjusted to meet the predetermined X-ray reference received dose,
wherein the at least one candidate region comprises an overexposed region and/or an underexposed region; and
add and synthesize at least one enhanced image corresponding to (i) the at least one candidate region, or (ii) the at least one candidate region and the target image, to obtain an X-ray image of the target region
